(19) United States
(12) Patent Application Publication
(10) Pub. No.: US 2024/0366731 A1
(43) Pub. Date: Nov. 7, 2024

(54) ORAL ALGAL OIL BASED GASTRO-INTESTINAL TRACT PERMEABLE PEPTIDE COMPOSITION

(71) Applicant: CELAGENEX RESEARCH (INDIA) PVT. LTD., Thane(West), Maharashtra (IN)

(72) Inventors: Rajaram SAMANT, Thane West (IN); Rajendra Prasad TONGRA, Jaipur (IN)

(73) Assignee: CELAGENEX RESEARCH (INDIA) PVT. LTD., Thane, Maharashtra (IN)

(21) Appl. No.: 18/683,214

(22) PCT Filed: Aug. 10, 2022
(86) PCT No.: PCT/IN2022/050717

§ 371 Date: Feb. 12, 2024

(30) Foreign Application Priority Data
Aug. 12, 2021 (IN) 202121006090

(51) Int. Cl. A61K 38/28; A61K 9/00; A61K 31/202; A61K 38/22; A61K 38/27; A61K 38/30; A61K 38/55; A61K 47/18; A61K 47/44; A61P 3/10

(52) U.S. Cl. CPC A61K 38/28; A61K 9/0053; A61K 31/202; A61K 38/2264; A61K 38/27; A61K 38/30; A61K 38/55; A61K 47/183; A61K 47/44; A61P 3/10

(57) ABSTRACT

The invention disclosed herein related to an oral, algal oil-based, gastro-intestinal tract permeable peptide composition. Particularly, the invention relates to the oral, water in algal oil-based, peptide compositions for the treatment of glucose metabolic disorders comprising peptides that generally degraded in GIT, a protease inhibitor and algal oil enriched with stabilized DHA along with pharmaceutically acceptable excipients, wherein protease inhibitor forms stoichiometric complex with the protease active site with activity of 5000 to 10,000 BAEE units per mg protein.

### TECHNICAL FIELD

The present invention relates to an oral, algal oil-based, gastro-intestinal tract (GIT) permeable peptide composition. Particularly, the invention relates to oral, algal oil-based peptide compositions for the treatment of metabolic disorders comprising peptides that are generally degraded in the GIT, a protease inhibitor, and a chelating agent in the presence of water in an algal oil along with pharmaceutically acceptable excipients. Particularly, the composition of the present invention is useful for treating glucose metabolism disorders.

### BACKGROUND OF THE INVENTION

Metabolic disorders (MDs) are characterized by the inability of the body to properly utilize and/or store energy. Metabolic disorder is a collection of cardio metabolic risk factors that include obesity, insulin resistance, hypertension, dyslipidemia, cognitive decline including decreased mental flexibility and memory deficits. Metabolic disorders are a cluster of conditions that occur together which include increasing risk of heart disease and stroke. Metabolic disorders also include increased blood pressure, high blood sugar, excess body fat around the waist and abnormal cholesterol or triglyceride levels. Metabolic disorders are increasingly common and up to one-third of the population suffers from them. Metabolic disorders could lead to serious health problems in the future, if not diagnosed properly in time. Metabolic disorders are mainly caused due to defective processing or transport of amino acids, fatty acids, sugars, and metals. More particularly, it is an enzyme deficiency leading to the disruption of normal bodily metabolism.

Diabetes mellitus (DM), commonly known as diabetes, is a metabolic disease that causes high blood sugar. The hormone insulin moves sugar from the blood into body cells to be stored or used for energy.

The global diabetes prevalence in 2019 was estimated to be 9.3% (463 million people), which will rise to 10.2% (578 million) by 2030 and 10.9% (700 million) by 2045. The prevalence is higher in urban (10.8%), than rural (7.2%) areas, and in high-income (10.4%) than low-income countries (4.0%). One in two (50.1%) people living with diabetes do not know that they have diabetes. The global prevalence of impaired glucose tolerance was estimated to be 7.5% (374 million) in 2019 and is projected to reach 8.0% (454 million) by 2030 and 8.6% (548 million) by 2045. [Diabetes research clinical practice 157, 107843, 2019]

Diabetes affects approximately 30.3 million people (9.4% of the population) in the United States, while another estimated 84.1 million people have prediabetes and do not know it. An estimated 7.2 million people in the United States have diabetes and do not even know it. In the past few years, diabetes has been the 7^{th} leading cause of death listed on death certificates in the United States.

As on date, most of the biomolecules are administered through the parental route of administration which has very low patient compliance. Repeated administration of a parenteral formulation causes lipoatrophy or lipohypertrophy. There are other adverse effects such as peripheral hyperinsulinemia, peripheral hypertension, the development of atherosclerosis, cancer, hypoglycaemia, and other adverse metabolic effects.

It is generally known that major biomolecules have low oral activity because of the way these substances are absorbed from the digestive system. Pharmacological studies indicate that biomolecules are absorbed from the intestines and carried by the blood through the portal system to the liver where they are rapidly inactivated by enzymatic reactions.

Particularly, the oral route carries biomolecules such as insulin directly to the liver through portal circulation, which is what also occurs in non-diabetic individuals. Adequate levels of insulin in the portal circulation are associated with a rapid and significant lowering of plasma glucose and haemoglobin A1c levels. Moreover, oral administration reduces systemic insulin exposure, and peripheral hyperinsulinemia.

Several attempts have been made to overcome the above obstacles by using approaches such as liposome delivery (U.S. Pat. No. 4,356,167), lipid coating (U.S. Pat. No. 4,849,227, WO8705505), use of peptidase inhibitors (U.S. Pat. No. 4,579,730), prodrugs, targeted microspheres (WO88/01213), coacervate systems (U.S. Pat. No. 4,849,405) or, lately, emulsion formulations. However, the prior art documents identified above have major disadvantages such as pH-sensitivity, promotion of unwanted expedited release, disaggregation, poor entrapment efficiency and poor stability in biological fluids.

Proteases, also called peptidases or proteinases, are enzymes that perform proteolysis. Proteolysis is one of the most important biological reactions. Proteolytic activity has been attributed to a class of enzymes called proteases. Based on the mechanism of action, proteases are classified as either serine, cysteine, or threonine proteases (amino-terminal nucleophile hydrolases), or as aspartic, metallo and glutamic proteases (with glutamic proteases being the only subtype not found in mammals so far).

Proteolytic enzymes help break down and digest protein. They are found in the body, as well as in certain foods and dietary supplements. Proteolytic enzyme supplements have recently grown popular due to health benefits. Bromelain, papain, pancreatin, trypsin and chymotrypsin are proteolytic enzymes that are commonly added to proteolytic supplement blends.

A trypsin inhibitor (TI) is a protein and a type of serine protease inhibitor (serpin) that reduces the biological activity of trypsin by controlling the activation and catalytic reactions of proteins. Trypsin is an enzyme involved in the breakdown of many different proteins, primarily as part of digestion in humans and other animals such as monogastric and young ruminants. When a trypsin inhibitor is consumed, it acts as an irreversible and competitive substrate.

There are various studies going on to develop an oral formulation of peptides with protease inhibitor. Some glucagon-like peptide-1 (GLP-1) receptor agonists like semaglutide available in oral dosage form frequently exhibit side effects such as risk of thyroid C-cell tumors, diabetic retinopathy complications, pancreatitis, acute kidney injury, serious hypersensitivity reactions in patients.

Certain compositions use fish oil comprising an omega-3 fatty acid. However, use of fish oil has well known disadvantages. Taking more than 3 grams per day might keep blood from clotting and increase the chances of bleeding. Fish oil can cause side effects including belching, bad breath, heartburn, nausea, loose stools, rash, nosebleeds, and high blood sugar. Fish oil accumulates toxins such as mercury, dioxins, and polychlorinated biphenyls (PCBs), and spoiled fish oil may produce peroxides. Although fish oil is the richest source of essential Omega-3 fatty acids EPA and DHA, the fishy odour, unstable nature, low purity, delivery form as capsules are major limitations of its use.

Krill oil (KO) is rich in polyunsaturated fatty acids (PUFAs), especially eicosapentaenoic acid (EPA) and docosahexaenoic acid (DHA), which have many health benefits. However, the oil is unstable owing to its high degree of unsaturation. The oil was microencapsulated using maltodextrin, saponin and sodium cascinate and rosemary oil (RO) extract or sodium ascorbate. [Food Hydrocolloids for Health Volume 1, 2021, 100031]

WO90/03164 discloses the use of oral proteinaceous compositions comprising oil/water emulsions which form chylomicra or deliver chylomicra to absorption sites in the gastrointestinal tracts. The compositions of WO'164 have drawbacks since oil/water emulsions are known in the art to have stability problems and involve denaturation of proteinaceous material due to heat and shear involved in the process.

U.S. Pat. No. 8,088,734B2 discloses enhancing bioavailability of peptide like salmon calcitonin to be administered orally is enhanced by acyl carnitines. WO1991004743A1 discloses a method for producing a stable preparation of a pharmaceutical peptide comprising stabilizing a solution of peptide in a physiologically acceptable aqueous buffer. More particularly, the invention relates to a formulation for stabilizing thymopentin in a low concentration. U.S. Pat. No. 6,034,054A provides a monomeric insulin formulation stabilized against aggregation in which the buffering agent is either tromethamine or arginine.

With respect to safety and convenience considerations, oral delivery satisfies the physiologic mode for biomolecules. To boost medication compliance, there is an immediate need to develop an oral delivery system for biomolecules which are site specific with accurate dosing, causing the biomolecule being administered to perform natural function more consistently, efficiently, and expediently. Also, the oral administration may avoid the cumbersome and expensive parenteral administration process with improved bioavailability.

Particularly the gastro-intestinal tract has many physiological barriers which prevent optimal delivery of oral peptide. Physiological function of the gut enzymes is to break large “active” proteins into smaller “inactive” amino acids so that they can overcome the second absorption barrier “tight epithelium” in the gastro-intestinal tract.

These two essential barriers have been created by Mother Nature to prevent body from potentially dangerous proteins. Researchers are trying to selectively break this natural defence mechanism so that helpful large “active protein” drugs can cross this barrier and produce desired pharmacological effects. Therefore, the need arises to co-administer the active proteins with enzyme inhibitors that improves the GIT absorption of protein for better efficacy.

In the current scenario, there is a need for an alternate source of oil enriched with omega-3 fatty acid preferably docosahexaenoic acid (DHA). It has been observed that docosahexaenoic acid alters the permeability of intestinal brush border membrane, making the intestinal brush border membrane more permeable to GIT degradable peptides, thereby enhancing intestinal peptide absorption. An emulsion incorporating highly purified long-chain polyunsaturated fatty acid, especially docosahexaenoic acid, has the potential of becoming the formulation for enteral delivery of peptides without intestinal wall damage.

Algal oil is a plant-based source of EPA and DHA, two omega-3 fats that are essential for human health. It may provide a great plant-based alternative if one doesn't cat seafood or can't tolerate fish oil. Even with advances in micro-encapsulation and other stabilization and oxidation inhibiting technologies, stability is still an issue with some omega-3-oils.

Moreover, stability will always be an issue for oils rich in eicosatetraenoic acid (EPA) and docosahexaenoic acid (DHA) as polyunsaturated fatty acids are very sensitive by nature to oxidation, which causes the fast degradation of volatile compounds. Hence it needs to be protected from oxidation.

By knowing the current scenario with regards to use of DHA, the present inventors have performed rigorous experiments and come up with a new vegan source enriched with docosahexaenoic acid, wherein this docosahexaenoic acid exhibits high oxidative stability and enhances oil-solubility and stability of the biomolecule, particularly GIT degraded peptides.

### OBJECTIVE OF THE INVENTION

The primary objective of the present invention is to provide an oral composition of biomolecules for treatment of metabolic disorder.

Another objective of the present invention is to provide novel drug delivery system for enhancing bioavailability of protein and peptide.

Another objective of the present invention is to provide a composition of biomolecules for oral administration which is therapeutically effective and improves patient compliance.

Yet another objective of the present invention is to provide eco-friendly approach for enhancing bioavailability of peptides by protecting them from degradation by intestinal enzymes.

A further objective of the present invention is to provide a therapeutically effective, novel, oral delivery system of biomolecules that are generally parenterally administered.

Another objective of the present invention is to provide a cost effective, non-toxic composition of a biomolecule for oral administration that facilitates transportation of biomolecule in a therapeutically effective amount for the treatment of metabolic disorder.

### SUMMARY OF THE INVENTION

To meet the above objectives, the inventors of the present invention carried out thorough experiments to establish significant therapeutic effects of the active ingredients or biomolecules or biological material or peptides or fatty acids or nutrients present in the composition for improving metabolic function in a subject in need thereof in a safer way. In an aspect, the invention relates to bioactive composition comprising therapeutically active nutrients along with pharmaceutically acceptable carriers for treating metabolic disorders. In another aspect, the invention provides bioactive composition comprising oral, algal oil based, GIT permeable composition for the treatment of metabolic disorders. In yet another aspect, the invention provides bioactive composition comprising biomolecules, a protease inhibitor, and a chelating agent in presence of an algal oil, along with pharmaceutically acceptable excipients.

In a particular aspect, the invention provides an oral algal oil based gastrointestinal tract permeable biomolecular composition for the treatment of diabetes mellitus which comprises a peptide, a protease inhibitor and ethylenediaminetetraacetic acid in presence of algal oil enriched with docosahexaenoic acid along with pharmaceutically acceptable excipients.

In another aspect, the invention provides an oral algal oil-based gastrointestinal tract permeable bio-molecular composition comprising peptide hormone in a range of 1-50 mg [10 IU to 2000 IU], soybean trypsin inhibitor in a range of 1-500 mg, ethylenediaminetetraacetic acid in a range of 1-500 mg, algal oil enriched with docosahexaenoic acid in a range of 1 to 10 ml, along with pharmaceutically acceptable excipients.

In yet another aspect, the invention discloses an oral algal oil-based gastrointestinal tract permeable bio-molecular composition for treating metabolic disorders such as acid-base imbalance, metabolic brain diseases, disorders of calcium metabolism, DNA repair-deficiency disorders, glucose metabolism disorders, hyperlactatemia, iron metabolism disorders, lipid metabolism disorders, malabsorption syndromes, metabolic syndrome X, inborn error of metabolism, mitochondrial diseases, phosphorus metabolism disorders, porphyrias, proteostasis deficiencies, metabolic skin diseases, wasting syndrome, and water-electrolyte imbalance. Particularly, the composition of the present invention is useful for treating glucose metabolism disorders such as diabetes mellitus and fatty liver disease.

### ABBREVIATIONS

MD: Metabolic disorder
DM: Diabetes mellitus
DHA: Docosahexaenoic acid
EPA: Eicosapentaenoic Acid
EDTA: Ethylenediaminetetraacetic acid
SBTI: Soybean trypsin inhibitor
DNA: Deoxyribonucleic acid
PCB: Polychlorinated biphenyl
NFLD: Non-alcoholic fatty liver disease
RNA: Ribonucleic acid
GIT: Gastrointestinal Tract
HbA1c: Haemoglobin A1c
FPG: Fasting plasma glucose
BAEE: N α-benzoyl-L-arginine ethyl ester

### BRIEF DESCRIPTION OF DRAWINGS

FIG. 1 illustrates A1c (HbA1c) % and FPG (mg/dl) level of placebo, reference standard (sitagliptin-100 mg) and composition 3-(oral insulin 8 mg).

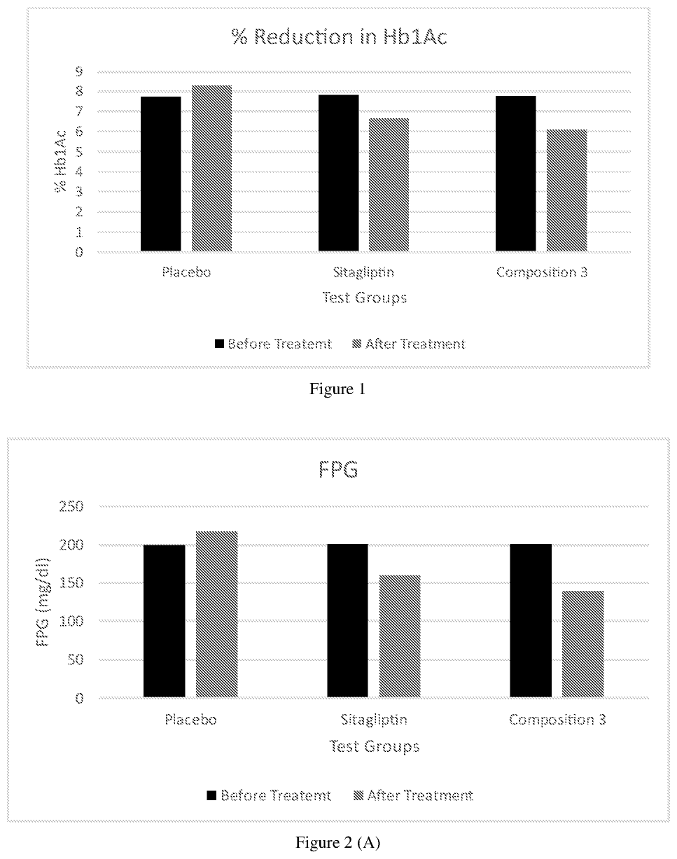

FIG. 2 illustrates the cleavage site of insulin by proteases. A: site of α-chymotrypsin; B: sites of trypsin.

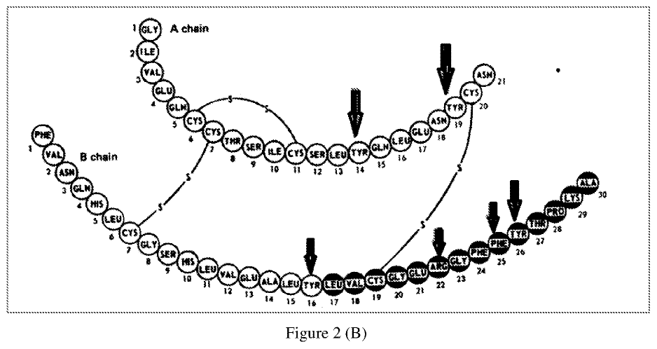

### DETAILED DESCRIPTION OF THE INVENTION

The invention will now be described in detail in connection with certain preferred and optional embodiments, so that various aspects thereof may be more fully interpreted and comprehended. However, any skilled person or artisan will appreciate the extent to which such embodiments could be generalized in practice.

All modifications and substitutions that come within the meaning of the description and the range of their legal equivalents are to be embraced within their scope. A description using the transition “comprising” allows the inclusion of other elements to be within the scope of the invention.

It is further to be understood that all terminology used herein is for the purpose of describing particular embodiments only and is not intended to be limiting in any manner or scope. Unless defined otherwise, all technical and scientific expressions used herein have the same meaning as commonly understood by one of ordinary skill in the art to which embodiments of the invention pertain. In describing and claiming the embodiments of the present invention, the following terminology will be used in accordance with the definitions set out below which are known in the state of art.

The singular forms “a,” “an,” and “the” include plural reference unless the context clearly dictates otherwise. Also, the term ‘composition’ does not limit the scope of the invention for multiple compositions that can be illustrated for best mode of the invention.

The term “pharmaceutically/nutraceutically acceptable salt,” as used herein, represents those salts which are within the scope of sound medical judgment and suitable for use in contact with the tissues of humans and animals without undue toxicity, irritation, allergic response and the like and are commensurate with a reasonable benefit/risk ratio. Particularly, the term “pharmaceutically-acceptable salts” refers to relatively non-toxic, inorganic, and organic acid addition salts of compounds, amino acid salts, sugar-based salts, alkali, or alkaline earth metal salts, as well as solvates, co-crystals, polymorphs, and the like of the salts.

The term ‘enriched’ refers to an increased quantity or concentration or proportion of a desirable ingredient in the sample to get improved therapeutic effect.

The term ‘Diabetes Mellitus (DM)’ also known as diabetes, is a group of metabolic disorders characterized by a high blood sugar level over a prolonged period. The symptoms often include frequent urination, increased thirst, and increased appetite. The most common types of diabetes are type 1, type 2, and gestational diabetes. In type 1 diabetes, the human body does not make insulin. The immune system attacks and destroys the cells in pancreas that make insulin. In type 2 diabetes, human body does not make or use insulin well. Gestational diabetes develops in some women when they are pregnant.

Emulsions are colloidal systems consisting of two liquid phases, oil and water, one of which is dispersed into the other. Water-in-oil (W/O) emulsions consist of an aqueous phase dispersed, in the form of small droplets, into a continuous oil phase.

Water-in-oil (W/O) emulsions have high potential for several industrial areas as delivery systems of hydrophilic compounds. In general, they are less studied than oil-in-water (O/W) systems, namely in what concerns the so-called fluid systems, partly due to problems of instability.

Water in oil emulsions are colloidal systems having water droplets dispersed throughout the oil. Therefore, oil acts as the continuous phase of this colloid while water is the dispersed phase. Oil does not mix with water under normal conditions. But with proper mixing and by using stabilizing agents, it can obtain oil in water emulsion. The effectiveness of this system enhances with a small size of dispersed oil droplets. It increases the bioavailability of the present composition, and it also increases the shelf life of the composition.

In a preferred embodiment, the invention provides an oral algal oil-based GIT permeable composition for the treatment of metabolic disorders comprising biomolecules, a protease inhibitor and ethylenediaminetetraacetic acid in the presence of algal oil along with pharmaceutically acceptable excipients.

In another preferred embodiment, the invention provides water in oil emulsions, wherein water droplets are dispersed throughout the algal oil.

Particularly the invention provides an oral water in algal oil-based GIT permeable composition for the treatment of metabolic disorders comprising biomolecules, a protease inhibitor and ethylenediaminetetraacetic acid along with pharmaceutically acceptable excipients.

In a particular embodiment, the biomolecular composition comprises one or more biomolecules selected from the group consisting of nucleic acid, amino acid, lipid, peptide, protein, hormone, antibody, enzyme, carbohydrate, DNA, RNA, polysaccharide, oligonucleotide, oligosaccharide, proteoglycans, and glycoprotein.

In another embodiment, the invention provides an oral composition wherein the biomolecule is a peptide hormone.

In a preferred embodiment, the biomolecule is peptide, particularly insulin, collagen, IGF-1, Testosterone undecanoate, leptin.

‘Insulin’ is a peptide hormone produced by beta cells of the pancreatic islets and is considered as the main anabolic hormone of the body. It regulates the metabolism of carbohydrates, fats, and protein by promoting the absorption of glucose from the blood into liver, fat, and skeletal muscle cells.

Insulin-like growth factor I (IGF-I) is a polypeptide hormone produced mainly by the liver in response to the endocrine GH stimulus. Insulin-like growth factor 1 (IGF-1), also called somatomedin C, is a hormone similar in molecular structure to insulin which plays an important role in childhood growth, and has anabolic effects in adults. IGF-I is partly responsible for systemic GH activities although it possesses a wide number of own properties (anabolic, antioxidant, anti-inflammatory and cytoprotective actions).

In another embodiment, the invention relates to the oral water in algal oil-based gastrointestinal tract permeable bio-molecular composition, wherein the protease inhibitor is selected from group consisting of from serine protease inhibitor, bovine pancreas trypsin inhibitor, basic pancreatic trypsin inhibitor, ovomucoid trypsin inhibitor, turkey ovomucoid trypsin inhibitor, soybean trypsin inhibitor, Kunitz trypsin Inhibitor, lima bean trypsin inhibitor and potato protease inhibitor.

‘Protease inhibitors’ are molecules that inhibit the function of a large group of naturally occurring enzymes called proteases. Protease inhibitors prevent proteases from splitting proteins into peptides. Proteases can either break specific peptide bonds or break down a complete peptide to amino acids (unlimited proteolysis).

In another preferred embodiment, the invention provides medicinal composition wherein protease inhibitor is soyabean trypsin inhibitor (STI) or serine protease inhibitors (SPI), preferably protease inhibitor is soyabean trypsin inhibitor (STI). Particularly enzymes such as pepsin, trypsin, chymotrypsin, carboxypeptidase and pancreatin break proteins such as peptide into amino acids.

In another embodiment, the invention provides an algal oil which is derived from marine or freshwater algae. The algal oil is contamination-free, non-toxic, and enriched with omega 3 fatty acid, particularly docosahexaenoic acid.

Algal oil, a type of oil derived from algae, stands out as one of the few vegan sources of both EPA and DHA. it's comparable to seafood regarding its medicinal availability of EPA and DHA. Particularly DHA from algal oil is especially beneficial to health.

Algal oil contains omega-3 and omega-9 fatty acids. These fatty acids can reduce inflammation (swelling), improve levels of some fats in the blood, and help with brain function. Algal oil rich in docosahexaenoic acid (DHA) is safe for most adults when taken by mouth. It has been used safely in studies for up to 4 years.

Algae oil is a plant-based oil which can be extracted from saltwater or freshwater micro-algae. It is sustainable and serves as a vegetarian option.

In another embodiment, the algal oil comprises enriched and stabilized docosahexaenoic acid, wherein stabilized DHA comprising specific combination of DHA and EDTA. Particularly DHA is stabilized by adding a specific quantity of Ethylenediaminetetraacetic acid EDTA. The stabilized DHA comprises DHA and EDTA or salts thereof in the weight ratio of 1:20 to 1:80.

‘Docosahexaenoic Acid’ also referred to as DHA, Doconexent or Cervonic acid, is chemically known as cis-docosa-4,7,10,13,16,19-hexaenoic acid. Docosahexaenoic acid (DHA) is an omega-3 fatty acid that is a primary structural component of the human brain, cerebral cortex, skin, and retina. In physiological literature, it has been given the name 22:6(n-3).

Ethylenediaminetetraacetic acid (EDTA) is also known as Edetic Acid which is the acid form of edetate. Ethylenediaminetetraacetic acid is a strong chelating agent that can remove ions required by digestive enzymes during proteolysis, thereby substantially reducing the proteolytic activity of the enzymes. The presence of ethylenediaminetetraacetic acid before the administration of peptide oil solution greatly enhances the therapeutic effect of peptide.

In a further embodiment, the invention provides an important role of docosahexaenoic acid in biomolecular absorption. Docosahexaenoic acid alters the permeability of intestinal brush border membrane which is more permeable to biomolecules. Docosahexaenoic acid is a safe carrier which enhances intestinal biomolecular absorption.

The stabilized docosahexaenoic acid has an enhancing effect on intestinal peptide absorption. Algal oil incorporating unsaturated fatty acids, particularly docosahexaenoic acid, is demonstrated to be a safe and useful carrier for enhancing peptide absorption via the intestinal tract.

In a preferred embodiment, the invention provides that the water in algal oil emulsion having water content of not more than 1% by weight of the total algal oil content in the composition.

In one preferred embodiment the invention provides the oral algal oil-based gastro-intestinal tract permeable peptide composition for the treatment of glucose metabolic disorders comprising combination of peptides which are degraded in the gastro-intestinal tract; protease inhibitor and algal oil enriched with stabilized docosahexaenoic acid along with pharmaceutically acceptable excipients

In preferred embodiment, the invention provides the oral algal oil based gastrointestinal tract permeable composition comprising therapeutically effective number of biomolecules, a protease inhibitor, and a chelating agent in presence of DHA enriched algal oil along with pharmaceutically acceptable excipients.

In another preferred embodiment, the invention provides medicinal composition for treating glucose metabolic disorders comprising therapeutically effective amount of biomolecules, a protease inhibitor, and a chelating agent in presence of DHA enriched algal oil along with pharmaceutically acceptable excipients.

In another preferred embodiment, the invention provides medicinal composition wherein the biomolecule is selected from the group consisting of peptide, peptide hormone, amino acid, nucleic acid, protein, hormone, antibody, enzyme, carbohydrate, DNA, RNA, polysaccharide, oligonucleotide, oligosaccharide, proteoglycans, and glycoprotein.

In another preferred embodiment, the invention provides medicinal composition wherein the peptide is hormone and active proteins.

In another preferred embodiment, the invention provides medicinal composition wherein the peptide hormone is selected from group consisting of adrenocorticotropic hormone (ACTH), amylin, angiotensin, atrial natriuretic peptide (ANP), calcitonin, cholecystokinin (CCK), gastrin, ghrelin, glucagon, growth hormone, follicle-stimulating hormone (FSH), insulin, leptin, luteinizing hormone (LH), melanocyte-stimulating hormone (MSH), oxytocin, parathyroid hormone (PTH), prolactin, renin, somatostatin, thyroid-stimulating hormone (TSH), thyrotropin, releasing hormone (TRH), vasopressin, also called arginine vasopressin (AVP) or anti-diuretic hormone (ADH), vasoactive intestinal peptide (VIP) and combination thereof.

In another preferred embodiment, the invention provides medicinal composition wherein the active proteins are selected from the group consisting of amino acids such as aspartic acid, asparagine, threonine, methionine, aspartic acid, asparagine, threonine, methionine, glutamic acid, proline, hydroxyproline, histidine, arginine, decarboxyl arginine, hydroxylysine, thyroxine, tryptophan, tyrosine, serine, and combination thereof.

In another preferred embodiment, the invention provides medicinal composition wherein the glucose metabolic disorders are selected from diabetes mellitus, glycosuria, hyperglycemia, hyperinsulinism, hypoglycemia.

In another preferred embodiment, the invention provides medicinal composition wherein the chelating agent is selected from ethylenediaminetetraacetic acid (EDTA), nitrilotriacetic acid, n-hydroxyethylethylenediaminetriacetic acid (HEDTA), simple organic acids like oxalic acid, malic acid, rubeanic acid and citric acid.

In another preferred embodiment, the invention provides medicinal composition wherein the algal oil is commercially available plant-based source of DHA and EPA, wherein DHA to EPA is present in the percentage ratio of 50%:50% to 95%:5%.

In one preferred embodiment, the invention provides medicinal composition wherein the invention provides an oral, water in algal oil based, gastrointestinal tract permeable composition comprising combination of peptide hormone and trypsin inhibitor and ethylenediaminetetraacetic acid (EDTA) in presence of algal oil, wherein algal oil is enriched with DHA.

In yet another preferred embodiment, the invention provides medicinal composition wherein the algal oil enriched with stabilized DHA comprising DHA 50-95% w/w and EPA 5-50% w/w. The stabilized DHA comprises DHA and EDTA or salts thereof in the weight ratio of 1:10 to 1:90.

Trypsin and α-Chymotrypsin are common protease in the intestine, insulin is degraded rapidly by α-chymotrypsin, and most of insulin was degraded after 60 min.

In one embodiment, the invention provides medicinal composition for enhancing the bioavailability of peptide hormone like insulin upon oral administration, wherein the composition comprises human insulin, trypsin inhibitor and algal oil enriched with stabilized DHA along with pharmaceutically acceptable excipients.

Particularly the effective amount of SBIT prevents the cleavages the specific site of the amino acid chain present in insulin molecule

Moreover, the present composition inhibits trypsin and a-chymotrypsin degradation. Particularly it inhibits cleavage site of trypsin and α-chymotrypsin. Tyrosine residue is the cleavage site of α-chymotrypsin. Arginine residue is one of the cleavage sites of trypsin.

In yet another embodiment, the oral composition, wherein the protease inhibitor is soybean trypsin inhibitor which inhibits peptide cleavage at Lysine, Tyrosine, Phenylalanine Arginine residue of trypsin and α-chymotrypsin sites.

α-Chymotrypsin is appeared to cleave initially at the carboxyl side of the B26-Tyrosine and A19-Tyrosine residues. Additionally, cleavage at B16-Tyrosine, B25-Phenylalanine, and A14-Tyrosine residues is also encountered rapidly. There are four Tyrosine residues (A14, A19, B16, B26) in the five cleavage sides. Further trypsin is cleaved insulin at the B29-Lysine and B22-Arginine residues. The degradation productions that cleaved in B29-Lysine by trypsin is insulin-like activity. The degradation is inhibited when insulin treated with trypsin inhibitor and stabilized DHA in algal oil with improved bioavailability of insulin.

In one another embodiment, the present invention provides the oral, water in algal oil-based, gastro-intestinal tract permeable peptide composition, wherein Trypsin Inhibitor is one of several protease inhibitors found in soybeans. Trypsin enzyme is inhibited by trypsin inhibitor at a molar ratio of 1:0.1 to 1:3, while chymotrypsin and plasmin are inhibited to a lesser extent.

Moreover 1 mg of trypsin inhibitor inhibits 0.1 to 3.5 mg trypsin with activity of more than 5000 BAEE units per mg protein. Particularly the present trypsin inhibitor activity is in the range of 5000 to 15000 BAEE units per mg protein, more particularly the present trypsin inhibitor activity is in the range of 5000 to 10,000 BAEE units per mg protein. Preferably, the present trypsin inhibitor activity is in the range of 7000 to 10,000 BAEE units per mg protein.

In further embodiment soybean trypsin inhibitor forms a 1:0.1 to 1:3 stoichiometric complexes with the protease active site. Particularly soybean trypsin inhibitor forms a 1:1 stoichiometric complex with the protease (trypsin and α-chymotrypsin) active site.

In one important embodiment the present invention provides, the oral algal oil based gastro intestinal tract permeable peptide composition for the treatment of glucose metabolic disorders comprising combination of peptides which are degraded in the gastro intestinal tract; protease inhibitor and algal oil enriched with stabilized docosahexaenoic acid along with pharmaceutically acceptable excipients, wherein protease inhibitor forms a 1:0.1 to 1:3 stoichiometric complex with the protease active site with activity of 5000 to 10,000 N-α-benzoyl-L-arginine ethyl ester [BAEE] units per mg protein.

The protease active sites are at Lysine, Tyrosine, Phenylalanine Arginine residue of trypsin and α-chymotrypsin sites. Trypsin hydrolyzes N-benzoyl-L-arginine ethyl ester (BAEE) at 253 nm and 25° C. This trypsin assay is performed to obtain a better activity value for trypsin, and following with the addition of the trypsin inhibitor, allow for the measurement of residual trypsin activity.

In a further embodiment, the invention provides an oral water in algal oil-based gastrointestinal tract permeable bio-molecular composition for treating metabolic disorders such as acid-base imbalance, metabolic brain diseases, disorders of calcium metabolism, DNA repair-deficiency disorders, glucose metabolism disorders, hyperlactatemia, iron metabolism disorders, lipid metabolism disorders, malabsorption syndromes, metabolic syndrome X, inborn error of metabolism, mitochondrial diseases, phosphorus metabolism disorders, porphyrias, proteostasis deficiencies, metabolic skin diseases, wasting syndrome and water-electrolyte imbalance. Particularly, the composition of present invention is useful for treating glucose metabolism disorders such as diabetes mellitus and fatty liver disease.

In yet another embodiment, the present invention provides an oral composition comprising a therapeutically effective amount of peptide or its derivative thereof, wherein peptide is present in a range of 1-500 mg of the total composition. In a preferred embodiment, peptide hormone and/or active protein is present in a range of 0.1-150 mg of the total composition.

In yet another embodiment, the present invention provides an oral composition comprising a therapeutically effective amount of protease inhibitor, wherein protease inhibitor is present in a range of 1-500 mg of the total composition. In a preferred embodiment, trypsin inhibitor is present in a range of 1-350 mg of the total composition.

In yet another embodiment, the present invention provides an oral composition comprising a therapeutically effective amount of ethylenediaminetetraacetic acid, wherein ethylenediaminetetraacetic acid is present in a range of 1-1000 mg of the total composition. In a preferred embodiment, Ethylenediaminetetraacetic acid [EDTA] is present in a range of 1-500 mg of the total composition.

In yet another embodiment, the present invention provides an oral composition comprising a therapeutically effective amount of stabilized docosahexaenoic acid, wherein stabilized docosahexaenoic acid is present in a range of 0.1-10 mg of the total weight of the oil. In a preferred embodiment, stabilized docosahexaenoic acid is present in a range of 0.1-5 mg of the total weight of the oil.

The term “therapeutically effective amount” denotes an amount that reduces the risk, potential, possibility or occurrence of a disease or disorder, or provides advanced alleviation, mitigation, and/or reduction or restoration or modulation, regulation of at least one indicator/biomarker (e.g., blood or serum CRP level), and/or minimizes at least one clinical symptom related to diabetes.

The term ‘subject in need thereof’ pertains to a subject preferably a mammal, more preferably a human suffering or suspected to be suffering from diabetes.

In the context of the present invention, the term “treatment” refers to alleviation, mitigation, prophylaxis, attenuation, management, regulation, modulation, control, minimization, lessening, decrease, down regulation, up regulation, moderation, inhibition, restoration, suppression, reversal, limitation, blocking, prevention, stabilization, amelioration, curing, or healing of metabolic disorders such as diabetes.

Notably, the present composition is non-hazardous, non-toxic, and safe for human consumption without any adverse effects. Therefore, the present medicinal composition is also used as preventive therapy/adjuvant therapy/add-on therapy/combination/adjunctive therapy in a subject in need thereof.

Certain compounds of the present invention exist in unsolvated forms as well as solvated forms, including hydrated forms. Further, some compounds of the present invention exist in multiple crystalline or amorphous forms (“polymorphs”). Compounds of the invention are formulated in geometric or, enatiomeric or stereoisomeric forms.

As used herein, the term “pharmaceutically acceptable carriers, diluents or excipients” is purported to mean, without limitation, any adjuvant, carrier, excipient, sweetening agent, diluents, preservative, dye/colorant, flavor enhancer, surfactant, wetting agent, dispersing agent, suspending agent, stabilizer, isotonic agent, solvent, emulsifier, or encapsulating agent, encapsulating polymeric delivery systems or polyethyleneglycol matrix, which is acceptable for use in the subject, preferably humans. Excipients also include antiadherents, antioxidants, binders, coatings, compression aids, disintegrants, dyes (colors), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, waters of hydration, salts.

In another embodiment, the invention relates to medicinal composition prepared in a manner well known in the pharmaceutical art, and administered by a variety of routes, depending upon whether local or systemic treatment is desired and upon the area to be treated. The preferable route of administration includes but is not limited to sublingual, rectal, topical, parenteral, nasal, or oral.

In some embodiment, the present medicinal composition is administered to a subject in need thereof, in the form which is suitable for oral use, such as a tablet, capsule (in the form of delayed release, extended release, sustained release, enteric coated release), hard gelatin capsules, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray.

In a further embodiment, the composition is formulated for parenteral use including intravenous, subcutaneous, intramuscular, intravascular, infusion, intraperitoneal, intracerebral, intracerebroventricular, or intradermal routes of administration.

The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose (in single or divided doses) ranges from about 10 mg per day to about 5000 mg per day, preferably about 50 mg per day to about 1500 mg per day.

The term “therapeutically effective amount” denotes an amount that reduces the risk, potential, possibility or occurrence of a disease or disorder, or provides advanced alleviation, mitigation, and/or reduction, restoration, modulation and/or minimization of at least one clinical symptom related to metabolic disorders.

The term ‘subject in need thereof’ pertains to a subject preferably a mammal, more preferably a human suffering or suspected to be suffering from metabolic disorders.

In the context of the present invention, the term “treatment” refers to alleviation, mitigation, prophylaxis, attenuation, management, regulation, modulation, control, minimization, lessening, decrease, down regulation, up regulation, moderation, curtailment, restriction, inhibition, restoration, suppression, reversal, limitation, blocking prevention, stabilization, amelioration, curing, or healing of glucose metabolic disorders.

Notably, the present composition is non-hazardous, non-toxic, and safe for human consumption without any severe adverse effects. The present medicinal composition is also used as preventive therapy/adjuvant therapy/add-on therapy/combination/adjunctive therapy in a subject in need thereof.

Certain compounds of the present invention exist in unsolvated forms as well as solvated forms, including hydrated forms. Further, some compounds of the present invention exist in multiple crystalline or amorphous forms (“polymorphs”). Compounds of the invention are formulated in geometric or, enantiomeric or stereoisomeric forms.

As used herein, the term “pharmaceutically acceptable carriers, diluents or excipients” is purported to mean, without limitation, any adjuvant, carrier, excipient, sweetening agent, diluents, preservative, dye/colorant, flavor enhancer, surfactant, wetting agent, dispersing agent, suspending agent, stabilizer, isotonic agent, solvent, emulsifier, or encapsulating agent, encapsulating polymeric delivery systems or polyethylene glycol matrix, which is acceptable for use in the subject, preferably humans. Excipients also include anti-adherents, antioxidants, binders, coatings, compression aids, disintegrants, dyes (colors), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, waters of hydration, or salts.

In another embodiment, the invention relates to a medicinal composition prepared in a manner well known in the pharmaceutical art, and administered by a variety of routes, depending upon whether local or systemic treatment is desired and upon the area to be treated. The preferable route of administration includes but is not limited to sublingual, rectal, topical, parenteral, nasal, or oral.

In yet another embodiment, the present-medicinal composition is administered to a subject in need thereof, in the form which is suitable for oral use, such as a tablet, capsule (in the form of delayed release, extended release, sustained release, enteric coated release); hard gelatin capsules, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray. In a further embodiment, the composition is formulated for parenteral use including intravenous, subcutaneous, intramuscular, intravascular, infusion, intraperitoneal, intracerebral, intracerebroventricular, or intradermal routes of administration.

The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose (in single or divided doses) ranges from about 1 mg per day to about 5000 mg per day, preferably about 100 mg per day to about 1500 mg per day.

Formulations of the present invention suitable for oral administration can be presented as discrete units such as capsules (e.g., soft-gel capsules), cachets or tablets each containing a predetermined amount of the active ingredient; as a powder or granules; as a solution or a suspension in an aqueous liquid or a non-aqueous liquid, syrup; or as an oil-in-water liquid emulsion or a water-in-oil liquid emulsion. The active ingredients can also be presented in the form of a bolus, electuary or paste, —bar, energy bars (candy bars), powder, or granule sachet.

Further, the present composition can be formulated in the form of age-appropriate paediatric oral dosage forms such as syrup, minitablets, chewable formulations, orodispersible films, or orodispersible tablets. It can also be prepared in the form of snack, chocolate bars or other confectionery food products.

In another embodiment, the composition of the present invention is non-toxic, cost effective, enriched with nutrients or biomolecules, and provides safeguard against problems associated with neurotransmission without any adverse/side effect.

In another embodiment of the invention, the diluents are selected from starches, hydrolyzed starches, partially pregelatinized starches, anhydrous lactose, cellulose powder, lactose monohydrate, sugar alcohols such as sorbitol, xylitol and mannitol, silicified microcrystalline cellulose, ammonium alginate, calcium carbonate, calcium lactate, dibasic calcium phosphate (anhydrous/dibasic dehydrate/tribasic), calcium silicate, calcium sulphate, cellulose acetate, corn starch, pregelatinized starch, dextrin, β-cyclodextrin, methylated-β-cyclodextrin, dextrates, dextrose, erythritol, ethyl cellulose, fructose, fumaric acid, glyceryl palmitostearate, magnesium carbonate, magnesium oxide, maltodextrin, maltose, medium-chain triglycerides, polydextrose, polymethacrylates, sodium alginate, sodium chloride, sterilizable maize, sucrose, sugar spheres, talc, trehalose, xylitol, vehicles like petrolatum, dimethyl sulfoxide and mineral oil or the like.

In some embodiment of the invention, the diluent in the composition/formulation is present in a range of 1% to 30% by weight of the total composition/formulation.

In yet another embodiment of the invention, the binder is selected from disaccharides such as sucrose, lactose, polysaccharides and their derivatives like starches, cellulose, or modified cellulose such as microcrystalline cellulose and cellulose ethers such as hydroxypropyl cellulose (HPC); hydroxypropyl methyl cellulose (HPMC); sugar alcohols such as xylitol, sorbitol, or mannitol; protein like gelatin; synthetic polymers such as polyvinylpyrrolidone (PVP), polyethylene glycol (PEG), starch, acacia, agar, alginic acid, calcium carbonate, calcium lactate, carbomers, carboxymethylcellulose sodium, carrageenan, cellulose acetate phthalate, chitosan, co-povidone, corn starch, pregelatinized starch, cottonseed oil, dextrates, dextrin, dextrose, ethyl cellulose, guar gum, hydrogenated vegetable oil, mineral oil, hydroxyethyl cellulose, hydroxymethyl cellulose, hydroxyl ethyl methyl cellulose, hydroxypropyl cellulose, inulin, cellulose, methyl cellulose, polyvinylpyrrolidone and polyethylene glycol, lactose, liquid glucose, hypromellose, magnesium aluminum silicate, maltodextrin, maltose, methyl-cellulose, microcrystalline cellulose, pectin, poloxamer, polydextrose, polymethacrylates, povidone, sodium alginate, stearic acid, sucrose, sunflower oil, various animal vegetable oils, and white soft paraffin, paraffin, flavorants, colorants and wax.

In further embodiment of the invention, the binder in the composition/formulation is present in a range of 0.1% to 40% by weight of the composition/formulation. In a preferred embodiment of the invention, the amount of binder is present in a range of 0.1% to 25% by weight of the composition/formulation.

In some embodiment, the antioxidant is selected from tocopherol (vitamin E), sesamol, guaiac resin, methionine, beta-carotene, lycopene, lutein, zeaxanthin, butylated hydroxyanisole (BHA), butylated hydroxytoluene (BHT), sodium ascorbate, sodium metabisulfite (SMB), 1-carnosine, propyl gallate (PG), tertiary butyl hydroquinone, cysteine (CYS), citric acid, tartaric acid, phosphoric acid, and ascorbic acid.

In some embodiment of the invention, the amount of antioxidant in the composition/formulation is present in the range of 0.1% to 10% by weight of the composition/formulation. In a preferred embodiment of the invention, the amount of antioxidant is present in a range of 0.1% to 5.0% by weight of the composition/formulation.

In another embodiment of the invention, the lubricant is selected from magnesium stearate, zinc stearate, calcium stearate, glycerin monostearate, glyceryl behenate, glyceryl palmitostearate, hydrogenated castor oil, hydrogenated vegetable oil, light mineral oil, magnesium lauryl sulphate, medium-chain triglycerides, mineral oil, myristic acid, palmitic acid, poloxamer, polyethylene glycol, sodium benzoate, sodium chloride, sodium lauryl sulphate, sodium stearyl fumarate, stearic acid, talc, potassium, or sodium benzoate or the like.

In some embodiment of the invention, the lubricant in the composition/formulation is present in a range of 0.1% to 10.0% by weight of the total composition/formulation.

In another embodiment of the invention, the solubilizing agent is selected from polysorbate 80, sodium lauryl sulphate, anionic emulsifying wax, non-ionic emulsifying wax, glyceryl monooleate, phospholipids, polyoxyethylene alkyl ethers, polyoxyethylene castor oil derivatives, polyoxyethylene sorbitan fatty acid esters, polyoxyethylene stearates, polyoxylglycerides, sorbitan esters, triethyl citrate, vitamin E, polyethylene glycol succinate, microcrystalline cellulose, carboxymethylcellulose sodium, diethanolamine, ethylene glycol palmitostearate, glycerin monostearate, hypromellose, hypromellose, acetate succinate, lecithin, polyethylene alkyl ethers, aluminum oxide, poly(methylvinyl ether/maleic anhydride), calcium carbonate, crospovidone, cyclodextrins, fructose, hydroxpropyl betadex, oleyl alcohol, povidone, benzalkonium chloride, benzethonium chloride, benzyl alcohol, benzyl benzoate, cetylpyridinium chloride, inulin, meglumine, poloxamer, pyrrolidone, sodium bicarbonate, starch, stearic acid, sulfobutylether beta cyclodextrin, tricaprylin, triolein, docusate sodium, glycine, alcohol, self-emulsifying glyceryl monooleate, cationic benzethonium chloride, cetrimide, xanthan gum, lauric acid, myristyl alcohol, butylparaben, ethylparaben, 15 methylparaben, propylparaben, sorbic acid or the like.

In another embodiment of the invention, the amount of solubilizing agent or surfactant in the composition/formulation ranges from 0.1% to 10% by weight of the composition/formulation. In a preferred embodiment of the invention, the solubilizing agent or surfactant is present in a range of 0.1% to 5.0% by weight of the composition/formulation.

In some embodiment of the invention, the glidant is selected from colloidal silicon dioxide, magnesium stearate, fumed silica (colloidal silicon dioxide), starch, talc, calcium phosphate tribasic, cellulose powdered, hydrophobic colloidal silica, magnesium oxide, zinc stearate, magnesium silicate, magnesium trisilicate, silicon dioxide or the like. In another embodiment of the invention, the glidant in the composition/formulation is present in a range of 0.1% to 5.0% by weight of the total composition/formulation.

In some embodiment of the invention, the stabilizers are selected from the group consisting of alginate, agar, carrageen, gelatin, guar gum, gum arabic, locust bean gum, pectin, starch, xanthan gum, trehalose and likewise.

In some embodiment of the invention, the stabilizer in the composition/formulation is present in a range of 0.1% to 8.0% by weight of the total composition/formulation. In a preferred embodiment of the invention, the amount of stabilizer is present in a range of 0.1% to 5.0% by weight of the composition/formulation.

In some embodiment of the invention, the plasticizers are added to coating formulations selected from the group propylene glycol, glycerol, glyceryl triacetate (triacetin), triethyl citrate, acetyl triethyl citrate, diethyl phthalate, acetylated monoglycerides, castor oil, mineral oil and like thereof.

In some embodiment of the invention, the plasticizer in the composition/formulation is present in a range of 0.1% to 5.0% by weight of the total composition/formulation.

In some embodiment of the invention, the solvent is selected from water, alcohol, isopropyl 10 alcohol, propylene glycol, mineral oil, benzyl alcohol, benzyl benzoate, flavored glycol, carbon dioxide, castor oil, corn oil (maize), cottonseed oil, dimethyl ether, albumin, dimethylacetamide, ethyl acetate, ethyl lactate, medium-chain triglycerides, methyl lactate, olive oil, peanut oil, polyethylene glycol, polyoxyl, castor oil, propylene carbonate, pyrrolidone, safflower oil, sesame oil, soybean oil, sunflower oil, water-miscible solvents, organic polar or non-polar solvents or mixtures thereof.

In a preferred embodiment of the invention, the solvent in the composition/formulation is used in a quantity sufficient to make the weight of the composition/formulation 100% by weight.

The additional additives include a polymer, a plasticizer, a sweetener, and a powdered flavor, a preservative, a colorant, a surfactant, and other excipients. The powdered flavor composition includes a flavourant associated with a solid carrier. Coating materials such as synthetic polymers, shellac, corn protein (zein) or other polysaccharides, gelatin, fatty acids, waxes, shellac, plastics, and plant fibers and like thereof are used.

In a preferred embodiment of the invention, the additives are used in a range of 1% to 20% w/w of unit dose. In a preferred embodiment of the invention, the number of additives is present in a range of 0.1% to 10% by weight of the composition/formulation.

In yet another embodiment, the invention provides peptide or protein composition along with pharmaceutical excipients, wherein the pharmaceutical excipients are selected from a diluent, a binder, a lubricant, a glidant, an additive, a surfactant, a stabilizer, or mixtures thereof.

In a preferred embodiment, the invention provides the novel and stable-composition wherein the pharmaceutically acceptable excipients are selected from a group consisting of the diluent is present in a range of 0.1 to 30%; the binder present is present in a range of 0.1 to 25%; the lubricant is present in a range of 0.1 to 10%; the glidant is present in a range of 0.1 to 5.0%; the additive is present in a range of 0.1 to 10%; the surfactant is present in a range of 0.1 to 5.0%; the stabilizer is present in a range of 0.1 to 5.0%; %; the antioxidant is present in a range of 0.1 to 5.0%; and the plasticizer is present in a range of 0.1 to 5.0%; by weight of total composition.

In another preferred embodiment, the present medicinal composition/formulation is formulated for oral administration. Specifically, the solid medicinal compositions, are in the form of tablets, capsules, pills, hard capsules filled with liquids or solids, soft capsules, sachets, powders, granules, suspensions, solutions, or modified release formulations. Formulations of the present invention suitable for oral administration are presented as discrete units such as capsules (e.g., soft-gel capsules, hard-gel capsule), cachets or tablets each containing a predetermined amount of the active ingredient; as a powder or granules; as a solution or a suspension in an aqueous liquid or a non-aqueous liquid, syrup; or as an oil-in-water liquid emulsion or a water-in-oil liquid emulsion.

In further embodiment compositions containing compounds of the present invention, can be conveniently presented in unit dosage form, and prepared by any of the methods well known in the art of pharmacy. Preferred unit dosage formulations are those containing an effective dose, or an appropriate fraction thereof, of the active ingredient, or a pharmaceutically acceptable salt thereof.

The magnitude of a prophylactic or therapeutic dose typically varies with the nature and severity of the condition to be treated and the route of administration. The dose, and perhaps the dose frequency, will also vary according to the age, body weight and response of the individual patient.

In general, the total daily dose (in single or divided doses) ranges from about 1 mg per day to about 2500 mg per day, preferably about 5 mg per day to about 1000 mg per day. In some embodiments, the total daily dose can range from about 5 mg to about 4000 mg per day, and preferably about 5 mg to about 2000 mg per day.

In certain embodiments, the invention provides the potent medicinal composition wherein the effective unit dose for an oral administration is formulated in a range of 1 to 1000 mg.

It is further recommended that children, patients over 60 years old, initially receive low doses and that the dosage be titrated based on individual physiological responses and/or pharmacokinetics. It can be necessary to use dosages outside these ranges in some cases, as will be apparent to those in the art. The present composition can be used as infant formula as well as adult formula by varying the concentration of active ingredients. Further, it is noted that the dietician or nutritionist or certified physician knows how and when to interrupt, adjust, or terminate therapy in conjunction with an individual patient's response.

The use of all examples, or exemplary language (e.g., such as) provided herein, is intended merely to better illuminate the invention, and does not pose a limitation on the scope of the invention unless otherwise claimed.

Various other examples of compositions and modifications or adaptations thereof can be devised by a person skilled in the art after reading the foregoing preferred embodiments without departing from the scope of the invention. All such further examples, modifications and adaptations are included within the scope of the invention.

It will be appreciated by those versed in the art that the present invention makes available novel and useful nutraceutical compositions and nutraceutical acceptable salts thereof, which have neuroprotective effects in several administration forms. Also, it will be understood by those with knowledge in the dietary supplement and nutraceutical art, that many embodiments of this invention may be made without departing from the scope of the invention, and the invention is not to be construed as limited, as it embraces all equivalents therein.

The invention may be further illustrated by the following examples, which are for illustrative purposes only and should not be construed as limiting the scope of the invention in anyway.

The present disclosure is therefore to be considered as in all respects illustrative and not restrictive, the scope of the invention being indicated by the appended claims and examples, and all changes or alterations which come within the ambit of equivalency are intended to be encompassed therein.

### EXAMPLES

Having described the basic aspects of the present invention, the following non-limiting examples illustrate specific embodiments thereof. Those skilled in the art will appreciate that many modifications may be made in the invention without changing the essence of invention.

### Example 1

Each capsule/tablet contains
Composition 1:

| | | |
|---|---|---|
| Peptide Hormone or Active | 1-100 | mg |
| Protein | | |
| Protease Inhibitor | 1-500 | mg |
| EDTA | 1-500 | mg |
| Algal oil | 1-10 | ml |

| | |
|---|---|
| DHA | ≥50% |
| EPA | ≤50% |
| Water in Oil emulsion | ≤1% |
| Excipient | 1-20% |

Composition 2:

| | | |
|---|---|---|
| Peptide Hormone | 1-100 | mg |
| Trypsin inhibitor (>5000 | 10-250 | mg |
| BAEE units) | | |
| EDTA | 10-200 | mg |

| | |
|---|---|
| Algal oil | 1 ml-5 ml |
| DHA | ≥50% |
| EPA | ≤50% |
| Water in Oil emulsion | ≤1% |
| Diluents | 1-10% |
| Binders | 0.1-5% |
| Glidants | 0.1-5% |
| Lubricants | 0.1-5% |
| Stabilizers | 0.1-10% |
| Additives | 1-10% |
| Antioxidant | 0.1-5% |
| Solvents | QS |

Composition 3:

| Insulin | 8 mg (231 IU) | |
|---|---|---|
| SBTI (7000 BAEE units) | 125 | mg |
| EDTA | 90 | mg |
| Algal oil | 2 | ml |
| DHA | 60% | |
| EPA | 40% | |
| Water in Oil emulsion | ≤1% | |
| Polymer of Acrylic and | 0.1-10 | mg |
| Methacrylic Acid Esters | | |
| Talc | 0.1-5 | mg |
| Polyethylene Glycol | 0.1-10 | mg |
| Sodium Benzoate | 0.1-10 | mg |
| Magnesium Stearate | 0.1-5 | mg |
| Phosphatidylcholine | 0.2-10 | mg |

Composition 4:

| Collagen | 50 | mg |
|---|---|---|
| SBTI (>5000 BAEE units) | 110 | mg |
| EDTA | 100 | mg |
| Algal oil | 3 | ml |
| DHA | 80% | |
| EPA | 20% | |
| Water in Oil emulsion | ≤1% | |
| Medium-chain triglycerides | 0.1-20 | mg |
| Croscarmellose sodium | 0.2-10 | mg |
| Polyvinylpyrrolidone | 0.1-20 | mg |
| Talc | 0.1-10 | mg |

| | |
|---|---|
| IPA | QS |

Composition 5:

| Human growth hormone HGH | 10-50 | mg |
|---|---|---|
| Trypsin inhibitor (>5000 BAEE | 150 | mg |
| units) | | |
| EDTA | 175 | mg |
| Algal oil | 5 | ml |
| DHA | 75% | |
| EPA | 25% | |
| Water in Oil emulsion | ≤1% | |
| Polymer of Acrylic and | 0.1-10 | mg |
| Methacrylic Acid Esters | | |
| Talc | 0.2-10 | mg |
| PVP K-30 | 0.1-10 | mg |

| | |
|---|---|
| IPA | QS |

Composition 6:

| IGF-I | 10 | mg |
|---|---|---|
| SBTI (>5000 BAEE units) | 125 | mg |
| EDTA | 90 | mg |
| Algal oil | 2 | ml |
| DHA | 70% | |
| EPA | 30% | |
| Water in Oil emulsion | ≤1% | |
| Ascorbic acid | 0.1-10 | mg |
| Talc | 0.1-10 | mg |
| Silicon dioxide | 0.1-10 | mg |
| Hydroxypropyl | 0.1-5 | mg |

| | |
|---|---|
| Methylcellulose | |
| IPA | QS |

Composition 7:

| Leptin | 10 | mg |
|---|---|---|
| STI (>5000 BAEE units) | 100 | mg |
| EDTA | 100 | mg |
| Algal oil | 2.5 | ml |
| DHA | 80% | |
| EPA | 20% | |
| Water in Oil emulsion | ≤1% | |
| Silicon dioxide | 1-5 | mg |
| Ascorbic acid | 0.1-5 | mg |
| Microcrystalline Cellulose | 0.1-10 | mg |
| Talc | 0.1-5 | mg |

Composition 8:

| Insulin | 15 mg (432 IU) | |
|---|---|---|
| SBTI (10000 BAEE units) | 150 | mg |
| EDTA | 125 | mg |
| Algal oil | 4 | ml |
| DHA | 85% | |
| EPA | 15% | |
| Water in Oil emulsion | ≤1% | |
| Sodium ascorbate | 0.1-5 | mg |
| Silicon dioxide | 0.1-10 | mg |
| Polymer of Acrylic and | 0.1-10 | mg |
| Methacrylic Acid Esters | | |
| Magnesium Stearate | 0.2-10 | mg |

Composition 9:

| Testosterone undecanoate | 80 | mg |
|---|---|---|
| SBTI (>5000 BAEE units) | 200 | mg |
| EDTA | 150 | mg |
| Algal oil | 4 | ml |
| DHA | 90% | |
| EPA | 10% | |
| Water in Oil emulsion | ≤1% | |
| Methacrylic Acid | 0.1-5 | mg |
| Talc | 0.1-5 | mg |
| Silicon oxide | 0.2-10 | mg |
| Ascorbic acid | 10 | mg |

| | |
|---|---|
| IPA | QS |

### Example 2—Animal Study

A study of Composition-3 vs Januvia (Sitagliptin) vs Placebo in adult patients with inadequate glycemic control
1. Composition-3—Oral insulin—8 mg
2. Reference—Januvia (Sitagliptin)—100 mg
3. Placebo

### Study Design and Participants

The pivotal study was involved 36 patients divided in to 3 groups of 12 each randomized to 1:1:1 into three cohorts dosed with 8 mg of Composition-3at night, Januvia 100 mg (Sitagliptin) at night and placebo at night.

Patient population-T2D adult patients above age of 21 years with inadequate glycemic control who were managing their condition with diet and metformin monotherapy.

### Inclusion Criteria:

HbA1c ≥7.5% on metformin ≥500 mg daily
At time of randomization, patients were treated for their diabetes by diet, exercise, and metformin (≥500 mg/day; any type and regimen). Patients were on a stable regimen of metformin (defined as the same metformin dose and type) for at least two weeks prior to entering the single-blind placebo run-in period.
Other anti-diabetic agents were not used for the two weeks prior to entering the placebo run-in period.
At Day-7 (Visit 3), all patients were HbA1c ≥7.5%
Body Mass Index between 25 and 40 kg/m2, inclusive.
Fasting blood glucose ≥126 mg/dL (8.3 mmol/L) prior to randomization at Day-7 (Visit 3).
Females of childbearing potential had a negative urine pregnancy test result at screening. A negative urine pregnancy test was obtained during Visit 2 and at Visit 4 (prior to randomization).
Males and females of childbearing potential used two methods of contraception (double barrier method), one of which must be an acceptable barrier method from the time of screening to the last study visit

### Exclusion Criteria:

Patients who meet any of the following criteria was not eligible for this study.
Presence of any clinically significant endocrine disease according to the Investigator (euthyroid patients on replacement therapy was included if the dosage of thyroxine is stable for at least six weeks prior to Screening Visit).
Clinical diagnosis of Type 1 diabetes.
Presence or history of cancer within the past five years except for adequately treated localized basal cell skin cancer or in situ uterine cervical cancer.
Laboratory abnormalities at screening including:
1. C-peptide <1.0 ng/mL.
2. Positive pregnancy test in females of childbearing potential (at screening and start of run-in period).
3. Abnormal serum thyrotropin (TSH) levels >1.5 times the upper limit of normal.
4. Positive test for hepatitis B surface antigen and/or hepatitis C antibody.
5. Positive test for HIV.
6. Serum Cr >1.4 mg/dl in males, >1.3 mg/dl in females.
7. Any relevant abnormality interfering with the efficacy or the safety assessments during study drug administration.
Use of the following medications:
1. History of use of insulin for greater than one week in the last six months and any use of insulin in the last six weeks prior to randomization.
2. Administration of anti-diabetic drugs other than metformin within four weeks prior to randomization visit. Administration of thyroid preparations or thyroxine within six weeks prior to screening visit. (Patients on stable thyroid replacement therapy for greater than 6 weeks may enter the study.)
Administration of systemic long-acting corticosteroids within two months or prolonged use (more than one week) of other systemic corticosteroids or inhaled corticosteroids within 30 days prior to screening visit.
Use of medications known to modify glucose metabolism or to decrease the ability to recover from hypoglycemia such as oral, parenteral, and inhaled steroids (as discussed above), beta blockers (except for beta blocker ophthalmic solutions for glaucoma or ocular hypertension), and immunosuppressive or immunomodulating agents.
History of tobacco or nicotine use more than two packs/day within ten weeks prior to screening.
Patient was on a weight loss program and was not in the maintenance phase, or patient that started any approved or non-approved weight loss medication within eight weeks prior to screening.
Pregnancy or breast-feeding.
Patient had a screening visit systolic blood pressure of ≥160 mm Hg or diastolic blood pressure of ≥100 mm Hg Patients were allowed to take BP medication as long as they have been on a stable dose for a period of four weeks prior to the screening visit.
Patient was, at the time of signing informed consent, a user of recreational or illicit drugs or had a recent history (within the last year) of drug or alcohol abuse or dependence. (Note: Alcohol abuse includes heavy alcohol intake as defined by >3 drinks per day or >14 drinks per week, or binge drinking).
Elevated liver enzymes (alanine transaminase (ALT), alanine aminotransferase (AST), alkaline phosphatase) greater than two times the upper limit of normal at screening.
Very high triglyceride level (>500 mg/dL) at screening.
Any clinically significant electrocardiogram (ECG) abnormality at screening or cardiovascular disease. Clinically significant cardiovascular disease will include:
1. History of stroke, transient ischemic attack, or myocardial infarction within six months prior to screening,
2. History of or currently have New York Heart Associate Class II-IV heart failure prior to screening, or
3. Uncontrolled hypertension defined as blood pressure ≥160 mmHg (systolic) or ≥100 mmHg (diastolic) at screening.

### Method:

The primary endpoint of the study was to compare the efficacy of Composition-3 to Sitagliptin & placebo in improving glycaemic control as assessed by A1c over a 04-week treatment period, with a secondary endpoint of comparing Composition-3 to Sitagliptin & placebo in change from baseline in FPG at week 04
1. Change from Baseline in Haemoglobin A1c (A1C) at Week 04 [Time Frame: Baseline and Week 04] A1C was measured as the percentage of glycosylated haemoglobin. Change in A1C following 04 weeks of therapy (i.e., A1C at Week 04 minus A1C at baseline)
2. Change from Baseline in Fasting Plasma Glucose (FPG) at Week 04 [Time Frame: Baseline and Week 04]
Change in FPG (before breakfast) following 04 weeks of therapy (i.e., FPG at Week 04 minus FPG at baseline)

Study Outcome:

| | | | | A1c (HbA1c) % | | FPG (mg/dl) | |
|---|---|---|---|---|---|---|---|
| Pt | | Age | | | Week | | Week |
| No | Sex | (yrs) | RANDOMISATION | Baseline | 04 | Baseline | 04 |
| 1 | M | 46 | Placebo | 7.8 | 8.1 | 200.38 | 211.06 |
| 2 | M | 48 | Sitagliptin | 7.6 | 6.6 | 193.26 | 152.06 |
| 3 | M | 47 | Composition-3 | 7.6 | 5.5 | 193.26 | 118.5 |
| 4 | M | 54 | Placebo | 7.6 | 8.2 | 193.26 | 214.62 |
| 5 | M | 52 | Sitagliptin | 7.7 | 6.4 | 196.82 | 158.54 |
| 6 | M | 52 | Composition-3 | 7.6 | 5.4 | 193.26 | 114.94 |
| 7 | F | 48 | Placebo | 8.1 | 8.4 | 211.06 | 221.74 |
| 8 | F | 51 | Sitagliptin | 7.9 | 6.6 | 203.94 | 155.06 |
| 9 | F | 55 | Composition-3 | 8 | 5.5 | 207.5 | 118.5 |
| 10 | M | 64 | Placebo | 7.8 | 8.3 | 200.38 | 218.18 |
| 11 | M | 62 | Sitagliptin | 8 | 6.8 | 207.5 | 159.18 |
| 12 | M | 59 | Composition-3 | 7.9 | 5.6 | 203.94 | 122.06 |
| 13 | F | 61 | Placebo | 7.6 | 7.9 | 193.26 | 203.94 |
| 14 | F | 62 | Sitagliptin | 7.6 | 6.5 | 193.26 | 156.1 |
| 15 | F | 60 | Composition-3 | 7.7 | 6.3 | 196.82 | 146.98 |
| 16 | M | 56 | Placebo | 7.6 | 8.2 | 193.26 | 214.62 |
| 17 | M | 56 | Sitagliptin | 7.7 | 6.4 | 196.82 | 150.54 |
| 18 | M | 55 | Composition-3 | 7.6 | 6.2 | 193.26 | 143.42 |
| 19 | M | 43 | Placebo | 7.7 | 8.4 | 196.82 | 221.74 |
| 20 | M | 46 | Sitagliptin | 7.8 | 6.5 | 200.38 | 158.1 |
| 21 | M | 45 | Composition-3 | 7.8 | 6.1 | 200.38 | 139.86 |
| 22 | F | 55 | Placebo | 7.7 | 8.3 | 196.82 | 218.18 |
| 23 | F | 58 | Sitagliptin | 7.8 | 6.6 | 200.38 | 166.66 |
| 24 | F | 59 | Composition-3 | 7.9 | 6.4 | 203.94 | 150.54 |
| 25 | F | 66 | Placebo | 7.6 | 8.1 | 193.26 | 211.06 |
| 26 | F | 68 | Sitagliptin | 7.7 | 6.6 | 196.82 | 157.66 |
| 27 | F | 64 | Composition-3 | 7.7 | 6.3 | 196.82 | 146.98 |
| 28 | F | 48 | Placebo | 7.7 | 8.4 | 196.82 | 221.74 |
| 29 | F | 49 | Sitagliptin | 7.8 | 6.8 | 200.38 | 164.78 |
| 30 | F | 54 | Composition-3 | 7.7 | 6.5 | 196.82 | 154.1 |
| 31 | M | 58 | Placebo | 7.8 | 8.5 | 200.38 | 225.3 |
| 32 | M | 59 | Sitagliptin | 7.9 | 6.8 | 203.94 | 164.78 |
| 33 | M | 60 | Composition-3 | 7.9 | 6.7 | 203.94 | 161.22 |
| 34 | M | 66 | Placebo | 8.1 | 8.6 | 211.06 | 228.86 |
| 35 | M | 63 | Sitagliptin | 8.2 | 7.3 | 214.62 | 182.58 |
| 36 | M | 65 | Composition-3 | 8.2 | 6.8 | 214.62 | 164.78 |

Observation:

| | | | % | | | % |
|---|---|---|---|---|---|---|
| | A1c (HbA1c) % | | Improvement | FPG (mg/dl) | | Improvement |
| Mean placebo (n = 12) | 7.76 | 8.28 | — | 198.90 | 217.59 | — |
| Mean sitagliptin | 7.81 | 6.66 | 14.7 | 200.68 | 160.53 | 20 |
| (n = 12) | | | | | | |
| Mean Composition-3 | 7.80 | 6.11 | 21.6 | 200.38 | 140.16 | 30 |
| (n = 12) | | | | | | |

Composition-3 significantly reduces blood sugar level subjects over the marketed anti-diabetic drug i.e., Sitagliptin. More particularly, sitagliptin reduces HbA1c level by 14.7% whereas composition 1 reduces HbA1c level by 22%. Fasting Plasma Glucose (FPG) level was reduced up to 20% by sitagliptin and up to 30% by Composition-1. Hence, it was proved that the Composition-1 as claimed in the present invention is effective in the treatment of diabetes with novel oral formulation without any severe side effects.

It may be noted that sitagliptin can cause serious side effects, including pancreatitis, which may be severe and lead to death. Sometimes it may cause increasing shortness of breath or trouble breathing and heart problems. The risk of getting low blood sugar is higher when taken along with sulfonylurea medicine or insulin.

1. An oral, algal oil-based, gastro-intestinal tract permeable peptide composition for treatment of glucose metabolic disorders, the composition comprising:
a combination of peptides which are degraded in the gastro-intestinal tract; protease inhibitor; and water in algal oil-enriched with stabilized docosahexaenoic acid (DHA) along with pharmaceutically acceptable excipients,
wherein the protease inhibitor forms 1:0.1 to 1:3 stoichiometric complex with protease active site with an activity of 5000 to 10,000 N-α-benzoyl-L-arginine ethyl ester (BAEE) units per mg protein.

2. The composition as claimed in claim 1, wherein the combination of peptides comprises peptide hormone and active protein.

3. The composition as claimed in claim 1, wherein the peptide hormone is selected from group adrenocorticotropic hormone (ACTH), amylin, angiotensin, atrial natriuretic peptide (ANP), calcitonin, cholecystokinin (CCK), gastrin, ghrelin, glucagon, growth hormone, follicle-stimulating hormone (FSH), insulin, leptin, luteinizing hormone (LH), melanocyte-stimulating hormone (MSH), oxytocin, parathyroid hormone (PTH), prolactin, renin, somatostatin, thyroid-stimulating hormone (TSH), thyrotropin, releasing hormone (TRH), vasopressin also called arginine vasopressin (AVP) or anti-diuretic hormone (ADH), and vasoactive intestinal peptide (VIP).

4. The composition as claimed in claim 1, wherein the active protein is selected from the group consisting of amino acids such as aspartic acid, asparagine, threonine, methionine, glutamic acid, proline, hydroxyproline, histidine, arginine, decarboxyl arginine, hydroxylysine, thyroxine, tryptophan, tyrosine, and serine.

5. The composition as claimed in claim 1, wherein the peptide hormone and the active protein is present in a range of 0.1 to 150 mg of the total composition.

6. The composition as claimed in claim 1, wherein the protease inhibitor is selected from serine protease inhibitor, bovine pancreas trypsin inhibitor, basic pancreatic trypsin inhibitor, ovomucoid trypsin inhibitor, turkey ovomucoid trypsin inhibitor, soybean trypsin inhibitor, Kunitz trypsin Inhibitor, lima bean trypsin inhibitor and potato protease inhibitor.

7. The composition as claimed in claim 6, wherein the protease inhibitor is soybean trypsin inhibitor.

8. The composition as claimed in claim 1, wherein the protease active sites are at lysine, tyrosine, phenylalanine, arginine residue of trypsin and α-chymotrypsin sites.

9. The composition as claimed in claim 1, wherein the protease inhibitor is present in a range of 1 to 500 mg of the total composition

10. The composition as claimed in claim 1, wherein the stabilized docosahexaenoic acid (DHA) comprises DHA and EDTA in a weight ratio of 1:10 to 1:90 of the total composition.

11. The composition as claimed in claim 1, wherein the stabilized docosahexaenoic acid (DHA) comprises DHA to EPA percentage ratio ranging from 50:50 to 95:5 of the total composition.

12. The composition as claimed in claim 1, wherein water content in the algal oil is not more than 1% by weight of total algal oil content of the composition.

13. The composition as claimed in claim 1, wherein the pharmaceutically acceptable excipients are selected from a group consisting of a diluent present in a range of 1 to 30%; a binder present in a range of 0.1 to 30%; an antioxidant present in the range of 0.1 to 10%; a lubricant present in a range of 0.1 to 5.0%; a glidant present in a range of 0.1 to 5.0%; an additive present in a range of 1 to 10%; a surfactant present in a range of 0.1 to 5.0%; a stabilizer present in a range of 0.1 to 5.0%; a plasticizer present in a range of 0.1 to 5.0%, by weight of the total composition.

14. The composition as claimed in claim 1, wherein an effective unit dose of the composition for oral administration is in a range of 1 to 1000 mg.

15. The composition as claimed in claim 1, wherein the glucose metabolism disorders comprise diabetes mellitus, glycosuria, hyperglycemia, hypoinsulinemia, hyperinsulinism, and hypoglycemia.
